(19) United States
(12) Patent Application Publication
(10) Pub. No.: US 2021/0401726 A1
(43) Pub. Date: Dec. 30, 2021

(54) Smuthcentz

(71) Applicant: Teresa Lynete Miller, Morgantown, WV (US)

(72) Inventor: Teresa Lynete Miller, Morgantown, WV (US)

(21) Appl. No.: 16/873,831

(22) Filed: Jun. 29, 2020

(51) Int. Cl. A61K 8/92; A61K 8/02; A61Q 19/00; C11C 5/00

(52) U.S. Cl. CPC A61K 8/922; A61K 8/0204; C11C 5/008; A61Q 19/00; C11C 5/002; A61K 8/925

(57) ABSTRACT

The claimed invention is lotion candle/melt. There is no wax in the product. They are safe if consumed by pets or children. They ae also safe for the environment being as they are not made from a kerosine by product. They are also safe to apply on your skin as a moisturizer due to the fact that they do not contain any wax product that is produced from kerosene. Child and pet safe they can be consumed with out harmful side effects being that they're made of all natural material if you are not allergic They contain no petroleum byproducts.

### BACKGROUND OF THE INVENTION

A lotion melt or lotion candle is similar to a traditional wax melt or candle. They are similar in the way that the product is used. But after that there are no similarities because A lotion melt and lotion candle it's made out of al materials that are safe if consumed by pets or children. You can also use it as a massage lotion.

You can also use it as a moisturizer for your skin without the harmful byproduct of a traditional wax candle or wax melts.

They also omit a fragrance in the air the same as a traditional wax candle or wax melts. But a lotion melt and lotion candles are not harmful and they are also not a fragrance their scent additive is flavoring.

It basically solves the problem of a traditional wax candle that contains petroleum products as it does not contain any and it burns the same way a traditional wax candle would

A lotion melt or lotion candle are made of raw materials derived rom a plant substance and combined with lanolin which comes from the wool of a sheep. Which can be omitted for the allergen free type.

They serve the same purpose as a traditional wax candle and wax melt. Except that they are also a moisturizer. And a massage lotion.

They burn the same way a traditional wax candle would. With a typical burn time that exceeds petroleum based candles.

The wax melts are also used the same way and produce the same effect as a traditional wax melt. But there is no harmful chemicals. They can be used on your skin and lips. They can also be used for massage.

The scent added also is safe for use on the skin because it is not a traditional fragrance it is a added flavoring

### DESCRIPTION

### 1. Field of the Invention

The present disclosure generally relates to a raw material met for use in a wax warmer, and more specifically, to a composition for lotion malts and candle when a traditional wick is introduced.

### 2. Description of the Background of the Invention

Typical wickless candle solutions include an electric warmer or a tea candle heated warmer and a plurality of lotion cubes designed to be heated therein. The lotion cubes are usually provided in a container that requires the consumer to take out and place the lotion cubes into the warmer. The lotion cubes are to be pushed up on bottom of packaging or turned up side down and dumped in hand

In other instances, a typical wickless candle solution includes a warmer and one or more candle melts. The lotion malt can be used as a moisturizer and message lotion with no wax added. They are also used as a moisturizer and air freshener without the effects of a typical wax candle being that they have no petroleum based product in them.

A lotion candle is poured into a container that has a wick attached to the bottom of it. After melting the raw materials together and pouring them in the container. They harden and are used the same way that a typical wax. Candle is. They are also used as a moisturizer and air freshener also a massage lotion without the effects of a typical wax candle being that they have no petroleum based product in them.

### BRIEF DESCRIPTION OF THE DRAWINGS

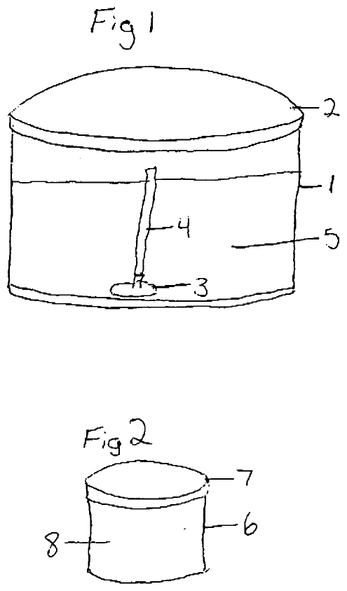

FIG. 1 shows an internal view of a 4 oz Smuthcentz container.
1 prior art Uline deep metal tin round 4 oz
2 prior art Uline window lid
3 prior art Candle wick durable sustainer wick tab
4 prior art braided wick cotton paper wick zinc wick
5 Smuthcentz

FIG. 2 shows a perspective view of a 1 oz Smuthcentz container.
6 prior art Uline plastic portion cup 1 oz
7 prior art Uline plastic portion lid

TEST 1.

Lotion Melt

The raw material melts are usually

Made of a raw material of A natural butler ie. ect. al. But as for example Shea butter. Also added Lanolin.

Further, typical raw material melts are designed to have a reasonably long shelf life so that the raw material melts can be produced, shipped, and positioned in a store for future sale. To make typical raw material melts, the components, are heated and blended together. Then the flavoring Is poured in. After blending, the molten composition is poured into the containers.

TEST 2

Lotion Melt

The raw material melts are usually

Made of a raw material of A natural butter ie. ect. al. But as for example Shea butter. Also added Lanolin, and almond oil. Further, typical raw material melts are designed to have a reasonably long shelf life so that the raw material melts can be produced, shipped, and positioned in a store for future sale. To make typical raw material melts, the components, are heated and blended together. Then the flavoring is poured in. After blending, the molten composition is poured into the containers.

TEST 3

Lotion Melt

The raw materiel melts are usually

Made of a raw material of A natural butter ie. ect. al. But as for example Shea butter. Also added Lanolin, and sesame oil depending on allergies. Further, typical raw material melts are designed to have a reasonably long shelf life so that the raw material melts can be produced, shipped, and positioned in a store for future sale. To make typical raw material melts, the components, are heated and blended together. Then the flavoring is poured in. After blending, the molten composition is poured into the containers.

TEST 4 LOTION MELT

The raw material melts are usually

Made of a raw material of A natural butter ie. ect. al. But as for example Shea butter, almond oil. Further, typical raw material melts are designed to have a reasonably long shelf life so that the row material melts can be produced, shipped, and positioned in a store for future sale. To make typical raw materdal melts, the components, are heated and blended together. Then the flavoring is poured in. After blending, the molten composition is poured into the containers. This is the allergen free type in case of allergic reactions to lanolin and sesame seed.

TEST 5

Lotion Candle

The raw material candles are usually Made of a raw material of A natural butter ie. ect. al. But as for example Shea butter. Also added Lanolin.

Further, typical raw material candles are designed to have a reasonably long shelf life so that the raw material candles can be produced, shipped, and positioned in a store for future sale. To make typical raw material candles, the components, are heated and blended together. Then the flavoring is poured in. After blending, the molten composition is poured into the containers. Which has a cotton wick added

TEST 6

Lotion Candle

The raw material candles are usually Made of a raw material of A natural butter ie. ect. al. But as for example Shea butter. Also added Lanolin, and almond oil. Further, typical raw material candle, are designed to have, reasonably long shelf life so that the raw material candles can be produced, shipped, and positioned in a store for future sale. To make typical raw material candles, the components, are heated and blended together. Then the flavoring is poured in After blending, the molten composition is poured into the containers.

Which has a cotton wick added

TEST 7

Lotion Candle

The raw material candles are usually Made of a raw material of A natural butter ie. ect. al. But as for example Shea butter. Also added Lanolin, and sesame oil depending on allergies. Further, typical raw material candles are designed to have a reasonably long shelf life so that the raw material candles can be produced, shipped, and positioned in a store for future sale. To make typical raw material candles, the components, are heated and blended together. Then the flavoring is poured in. After blending, the molten composition is poured into the containers. Which has a cotton wick added

TEST 8

Lotion Candle

The raw material candles are usually Made of a raw material of A natural butter ie. ect. al. But as for example Shea butter. Also added and almond oil. Further, typical raw material candles ae designed to have a reasonably long shelf life so that the raw material candles can be produced, shipped, and positioned in a store for future sale. To make typical raw material candles, the components, are heated and blended together. Then the flavoring is poured in. After blending, the molten composition is poured into the containers.

Which has a cotton wick added

This is the allergen free type in case of an allergic reactions to lanolin and sesame seed.

Specification

1.

A lotion candle includes between about eighty to ninety percent of. A raw materials of a natural butter ie. ect. Al. Shea butter given as example. 90 to 97 percent Shea to 3 percent of lanolin and about three percent sweet almond massage oil or sesame oil and about 10 percent to 40 percent by weight of flavoring, and between about the cornstarch does not have to be added and on occasion is not 10 percent of filler cornstarch and about 20 to 30 percent by weight of a steric acid.

2.

A lotion candle includes between about eighty to ninety percent of. A raw materials of a natural butter ie. ect. Al. Shea butter given as example. 90 to 97 percent Shea to about 3 percent sweet almond massage oi and about 10 percent to 40 percent by weight of flavoring, and between about the cornstarch does not have to be added and on occasion is not 10 percent of filer cornstarch and about 20 to 30 percent by weight of a steric acid.

3

A raw material melt includes between about eighty to ninety percent of. A raw materials of a natural butter ie. ect. Al. Shea butter given as example. 90 to 97 percent Shea to 3 percent of lanolin and about three percent sweet almond massage of or sesame all and about 10 percent to 40 percent by weight of flavoring, and between about the cornstarch does not have to be added and on occasion is not 10 percent of filer cornstarch and about 20 to 30 percent by weight of a steric acid.

4

A raw material melt includes between about eighty to ninety percent of. A raw materials of a natural butter ie. ect. Al. Shea butter given as example. 90 to 97 percent Shea to 3 percent about 3 percent sweet almond massage oil and about 10 percent to 40 percent by weight of flavoring, and between about the cornstarch does not have to be added and on occasion is not 10 percent of filler cornstarch and about 20 to 30 percent by weight of a steric acid.

What is claimed is:

1. A flavored lotion candle includes at least three different natural materials with one of the materials having one melting point and at least one of the other natural materials having a second higher melting point and the third material having a highest melting point: the natural material having the high melting is an oil almond, sesame, coconut, or lanolin ie. ect. Al.; the second natural material would be a butter coconut, Shea, mango, hemp. Ie ect. Al. may also be at a much higher concentration; the highest natural material having the highest melting point is a solid fatty acid obtained from animal or vegetable fat steric acid will also be at a much higher concentration than the fragrances and the other natural materials contained in the candle.

2. For the claimed invention according to claim 1; a lotion candle to burn, a heat source (commonly a naked flame from a match or lighter) is used to light the candle's wick, which melts and vaporizes a small amount of fuel (the lotion), once vaporized, the fuel combines with oxygen in the atmosphere to ignite and form a constant flame.

3. The claimed invention according to claim 1, is a lotion candle they are safe if consumed by pets or children, environmentally friendly being as they are not made from a kerosene byproduct, safe to apply on your skin as a moisturizer due to the fact that they do not contain any wax product that is made from kerosene.
